(19) United States
(12) Patent Application Publication
(10) Pub. No.: US 2022/0047566 A1
(43) Pub. Date: Feb. 17, 2022

(54) LONG ACTING IN-SITU FORMING/GELLING COMPOSITIONS

(71) Applicant: Humanwell Pharmaceutical US, Ballwin, MO (US)

(72) Inventors: Zhiwen Tang, Ballwin, MO (US); Jun Yang, Ballwin, MO (US); Baohua Yue, Ballwin, MO (US); Chang He, Ballwin, MO (US); Yunhua Li, Ballwin, MO (US); Vijay Kumar Nekkanti, Ballwin, MO (US)

(21) Appl. No.: 17/404,358

(22) Filed: Aug. 17, 2021

Related U.S. Application Data
(60) Provisional application No. 63/066,547, filed on Aug. 17, 2020.

(51) Int. Cl. A61K 31/445; A61K 9/06; A61K 31/167; A61K 31/485; A61K 31/415; A61K 31/5415; A61K 31/573; A61K 31/5377; A61K 31/438; A61K 31/4178; A61P 23/02; A61K 9/00

(52) U.S. Cl. CPC A61K 31/445; A61K 9/06; A61K 31/167; A61K 31/485; A61K 31/415; A61K 9/0019; A61K 31/573; A61K 31/5377; A61K 31/438; A61K 31/4178; A61P 23/02; A61K 31/5415

(57) ABSTRACT

The present invention provides sustained release formulations comprising one or more active pharmaceutical ingredient(s); at least one biocompatible polymer excipient; and at least one biocompatible solvent; methods for preparing the sustained release formulations, and methods for treating localized pain in a subject in need thereof.

### CROSS-REFERENCE TO RELATED APPLICATION

This application claims benefit of U.S. Provisional Patent Application No. 63/066,547, which was filed in the U.S. Patent and Trademark Office on Aug. 17, 2020, all of which is incorporated herein by reference in its entirety for all purposes.

### FIELD OF THE INVENTION

The present disclosure generally relates to sustained release formulations, methods for preparing the sustained release formulation, and methods of using the sustained release formulation where these sustained release formulations are in-situ forming-gelling formulations.

### BACKGROUND OF THE INVENTION

Sustained release drug delivery systems improve the safety and efficacy of drugs by optimizing their biopharmaceutical, pharmacokinetic and pharmacodynamics properties. Compared to conventional dosage forms, the sustained release drug delivery systems have several advantages such as improved patient compliance, steady-state drug levels, enhanced bioavailability, decreased side effects, lower healthcare costs. However, the development of sustained release drug delivery system is challenging due to the complex biological interactions and unique physicochemical properties of different drugs. Thus, there is still an unmet demand and market for long acting products in many therapeutic fields such as pain management, anti-viral, cancer therapy, CNS, etc.

The efficacy of local anesthetics usually lasts for hours, which is long enough to cover most surgical or invasive diagnostic process. However, after the surgical process, patients still suffer pain for much longer period. Increasing efficacy period by simply increasing the anesthetic dose may cause severe toxic effects. Current solution for treating this post-operative pain (POP) mainly relies on continuous administration of analgesics through different routes, such as repeated injection of short duration local anesthetic, local anesthetic pump, or patient controlled analgesia (PCA). Many of these methods are inconvenient and include the use of opioid drug. The use of opioid analgesia, especially through PCA, may raise severe safety concerns such as possible narcotic addiction, vomiting and respiratory depression. Thus, there is a great need of developing long acing analgesic product for this purpose. Extended release injectable formulations were developed to address the need by loading one or more analgesic ingredient to a sustained release formulation vehicle. The resulting complex injection formulation makes one-time administration for POP possible, as well as reducing the use of opioid drug (US 20130189349A1, U.S. Pat. No. 8,834,921B2, U.S. Pat. Nos. 9,668,974, 9,694,079, 5,244,678).

Different approaches such as biodegradable polymers, (US 20150182512A1, U.S. Pat. No. 9,694,079B2) and viscous oil formulations (U.S. Pat. No. 10,206,876B2), and liposomes (U.S. Pat. No. 8,834,921B2) have been developed as a vehicle to load analgesics and extend the release profile. Both opioids and non-opioids analgesics, such as morphine, bupivacaine, ropivacaine, and buprenorphine have been loaded into these novel extend release vehicles.

Exparel®, the first FDA approved long acting local anesthetic product in this field has been on market since 2012, utilizes multivesicular liposome as delivery vehicle for loading bupivacaine to achieve long acting anesthetic effect up to 72 hours. However, manufacture of multivesicular liposome product is challenging. The drug release and anesthesia efficacy duration of liposome product is also limited.

U.S. Pat. No. 10,213,510 describes a polymer formulation developed by Heron Therapeutics, Inc. Polyorthoester materials were used as vehicle for loading bupivacaine and meloxicam, a nonsteroidal anti-inflammatory drug (NSAID) to achieve long acting anesthetic effect up to 72 hr. The product, ZynRelief was approved for postoperative pain management and is formulated in a controlled-diffusion Biochronomer® polymer for consistent delivery of bupivacaine and meloxicam. The animal and human clinical trials proved that meloxicam is critical to extend the efficacy and it may cause an increased risk of serious cardiovascular side effects. This polymer has also been used in another marketed product SUSTOL, for extended release of Granisetron for chemotherapy-induced nausea and vomiting. However, this formulation has high viscosity and cannot be injected without adding viscosity reducing ingredient.

Durect developed SABER long acting platform using sucrose acetate isobutyrate (SAIB) and N-methyl pyrrolidone (NMP) solvents to dissolve drug substance (U.S. Pat. No. 8,153,149, Controlled delivery system). The product will form viscous gel matrix after injection and release the drug over extended period. Due to the concern of safety and efficacy, the product POSIMIR has been approved only for administration into the subacromial space under direct arthroscopic visualization.

U.S. Pat. No. 8,236,292B2 utilizes neutral diacyl lipid/tocopherol, phospholipid, and biocompatible low viscous organic solvent to dissolve or disperse active pharmaceutical ingredient to prepare a low viscous mixture with liquid crystalline phase structure. The mixture forms viscous gel when it comes in contact with aqueous and exhibits slow release of the drug. This FluidCrystal system can deliver both small molecules and biomolecules such as peptides (U.S. Pat. No. 8,865,021B2, Compositions of lipids and cationic peptides). Many products have been developed by Camurus using FluidCrystal technology. Similar long acting technology has also been reported by PainReform Ltd. for long acting local anesthesia purpose (U.S. Pat. No. 9,849,088, Depot formulations of a hydrophobic active ingredient and methods for preparation thereof). The proliposomal oil formulation forms liposomal structure after administration to achieve extended release of ropivacaine. The extended efficacy from these formulations have been reported but the benefit is limited.

Xaracoll is an FDA approved drug/device combination product to produce postsurgical local analgesia for up to 24 hours after inguinal hernia repair surgery. It uses collagen matrix to extend the release of bupivacaine in surgery site (USRE 47,826 Drug delivery device for providing local analgesia, local anesthesia or nerve blockage). However, the requirement of implant matrix in surgical site limited the application of Xaracoll.

Taiwan Liposome utilizes multi-lamellar liposome to load ropivacaine for postoperative pain management (WO 2020176568A1, Pharmaceutical compositions for use in treating pain). The clinical result demonstrated there is limited benefit compared to standard care using bupivacaine injection.

Lipocure and VirPax prepared a bupivacaine loaded liposome hydrogel by mixing multi-lamellar liposome with alginate hydrogel. The combination of multi-lamellar liposome and alginate hydrogel provides extended release of drug payload through dual long-acting mechanisms. However, the manufacturing process is complex and challenging.

PLGA is a biodegradable and biocompatible material. It has been widely used for fabricating the controlled release pharmaceutical products. The dosage forms include microsphere, in-situ forming, nanoparticles, etc. Alkermes used oil-in-water emulsion method to load drugs such as risperidone, naltrexone, etc. in PLGA microparticles (U.S. Pat. No. 5,792,477, Preparation of extended shelf-life biodegradable biocompatible microparticles containing a biologically active agent). After injected into body, the drug can be released for a long period from 2 weeks to several months. Liquidia developed PRINT technology to fabricate PLGA microparticles with designed shape and size which can be used to control the release of bupivacaine (U.S. Pat. No. 9,744,715, Method for producing patterned materials). Indivior developed an in-situ forming formulation to dissolve buprenorphine and PLGA in N-methyl-2-pyrrolidone (U.S. Pat. No. 10,198,218-Injectable flowable composition comprising buprenorphine). Once it's injected into the body, it forms PLGA gel matrix with buprenorphine trapped inside. The buprenorphine is slowly released from the PLGA gel matrix for up to one month. However, PLGA material will stay in the injection site for long period (2 weeks to several months) which is not ideal for applications require shorter than 1 week.

Amaca Thera developed a hydrogel drug delivery system using hyaluronic acid and methylcellulose. The high concentration of hyaluronic acid and methylcellulose makes the manufacture and clinical practice challenging due to high viscosity of the product.

Among the various complex formulation matrix material, Hyaluronic acid is an ideal candidate material due to its excellent biocompatibility and biodegradability. Hyaluronic acid is a negatively charged polysaccharide material, which naturally occurring in human body and is gradually degraded by Hyaluronidases. Lidocaine, ropivacaine, bupivacaine and other local anesthesia have been loaded into a hyaluronic acid containing matrix. To prepare an extended-release matrix, hyaluronic acid is often crosslinked to certain degree and dissolved in water or aqueous solution. However, hyaluronic acid formulation suffers the drawback of high viscosity that limits the design of formulation with extended-release performance. (U.S. Pat. No. 10,098,961B2 Hyaluronic acid composition, KR 102030508B1-Hyaluronic acid composition, KR 20140025117A Composition of anesthetic comprising hyaluronic acid, WO 2019121694A1 Injectable compositions of cross-linked hyaluronic acid and bupivacaine, and uses thereof, JP 433462062 A pharmaceutical product comprising a salt of hyaluronic acid with a local anesthetic.)

Besides the advantages mentioned above, there are still limitations and unmet needs in this field. Even though Exparel, the first marketed complex formulation product claims its efficacy lasts up to 72 hours, there is still unmet needs for longer efficacy in this field. Polymer formulations have more potential in achieving longer efficacy, but the viscosity of polymer formulation is usually very high which makes the administration difficult. The use of various other materials also raised safety concerns and unneglectable side effects that were observed during the clinical investigation. Hyaluronic acid, sodium hyaluronate, and cross-linked derivatives of hyaluronic acid are highly biocompatible materials that show promising application in this field, but the performance of hyaluronic acid, sodium hyaluronate, and cross-linked derivatives of hyaluronic acid needs to be improved by designing a suitable formulation. It would be desirable to have an improved formulation with low toxicity and high biocompatibility for long-acting local anesthetic effect and to ease the post-operative pain management and reduce the use of opioids drugs.

What is needed is a sustained release formulation using biocompatible excipients and solvents with dispersed/dissolved drug content that forms partial gelation with the polymer. The partial gelation polymer can be further hydrated to form an in-situ gel matrix after administration into body. The hydrated in-situ gel matrix provides the sustainable release of drug payload to surrounding tissue to achieve long acting local anesthetic effect.

### FIGURES

FIG. 1 represents the percentage of active pharmaceutical ingredients of two novel formulations versus time

FIG. 2A-2F are photographs which show the gelation of sodium hyaluronate in a dialysis bag and the release of the active pharmaceutical ingredient over a period of time in-vitro release study. FIG. 2A is a photograph after 0 minutes showing the gelation and active pharmaceutical ingredient in the in-vitro release study. FIG. 2B is a photograph after 1 hour showing the gelation and active pharmaceutical ingredient in the in-vitro release study. FIG. 2C is a photograph after 2 hours showing the gelation and active pharmaceutical ingredient in the in-vitro release study. FIG. 2D is a photograph after 4 hours showing the gelation and active pharmaceutical ingredient in the in-vitro release study. FIG. 2E is a photograph after 6 hours showing the gelation and active pharmaceutical ingredient in the in-vitro release study. FIG. 2F is a photograph after 24 hours showing the gelation and active pharmaceutical ingredient in the in-vitro release study. As the in-vitro release study progresses, the formulations became clearer and were clear at the end of the study.

FIG. 3A-3F are photographs which show another perspective of the gelation of sodium hyaluronate in a dialysis bag and the release of the active pharmaceutical ingredient over a period of time in-vitro release study. FIG. 3A is a photograph after 0 minutes showing the gelation and active pharmaceutical ingredient in the in-vitro release study. FIG. 3B is a photograph after 1 hour showing the gelation and active pharmaceutical ingredient in the in-vitro release study. FIG. 3C is a photograph after 2 hours showing the gelation and active pharmaceutical ingredient in the in-vitro release study. FIG. 3D is a photograph after 4 hours showing the gelation and active pharmaceutical ingredient in the in-vitro release study. FIG. 3E is a photograph after 6 hours showing the gelation and active pharmaceutical ingredient in the in-vitro release study. FIG. 3F is a photograph after 24 hours showing the gelation and active pharmaceutical ingredient in the in-vitro release study. As the in-vitro release study progresses, the formulations became clearer and were clear at the end of the study.

FIG. 4 shows a graph which demonstrates the in-vitro release of an active pharmaceutical ingredients in four formulations disclosed.

FIG. 5 shows the results of a rat sciatic block study of Formulation 1 and 2 versus direct administration of levobupivacaine HCl where the graph plots the response (pain) versus time. Formulations 1 and 2 show extended efficacy compared to levobupivacaine HCl sample demonstrating superior efficacy of the suspension formulations. The drug concentration difference in these two formulations didn't affect the efficacy significantly.

FIG. 6 shows the results of a rat sciatic block study of Formulation 3, 4 and 5 versus direct administration of bupivacaine HCl where the graph plots the response (pain) versus time. Formulation 3 show prolonged efficacy compared to bupivacaine HCl. The addition of betamethasone-21-acetate in Formulations 4 and 5 further improved the efficacy period.

FIG. 7 shows the results of a rat sciatic block study of Formulation 6 and 7 where the graph plots the response (pain) versus time. Both Formulation 6 and 7 showed similar efficacy period. The different amount of sodium hyaluronate used in these two formulations didn't affect the efficacy significantly in rat sciatic block model.

FIG. 8 shows the results of an animal study of mini pig skin incision model where the graph plots the response (pain) versus time comparing saline, levobupivacaine HCl, and Formulation 8. The mini pigs injected with saline could feel the pain after 30 min of surgery. As the effect of isoflurane anesthesia subsided, the saline group mini-pigs' response force dropped dramatically. The efficacy of levobupivacaine HCl injection can last for about 4 hours, which is similar to reported literature. The anesthesia efficacy of Formulation 8 lasted over 40-56 hours, which is significantly longer than levobupivacaine HCl.

### SUMMARY OF THE INVENTION

In one aspect, provided herein, are sustained release formulations. The sustained release formulations comprise: (a) one or more active pharmaceutical ingredient(s), (b) at least one biocompatible polymer excipient; and (c) at least one solvent; wherein one active pharmaceutical ingredient has a particle size distribution ranging from about 0.5 μm to about 100.0 μm. These formulations form an in-situ gel upon contact with water or physiological fluid.

In another aspect, provided herein, is a method of preparing a sustained release formulation, the method comprises contacting one or more active pharmaceutical ingredient(s), at least one biocompatible polymer excipient, and at least one solvent; wherein one of the active pharmaceutical ingredients has a particle size distribution ranging from about 0.5 μm to about 100.0 μm.

In still another aspect, provided herein, is method of treating localized pain in a subject in need, the method comprises locally administering the sustained release formulation as described above.

Other features and iterations of the invention are described in more detail below.

### DETAILED DESCRIPTION OF THE INVENTION

In one aspect, the present disclosure provides a sustained release formulation. The sustained release formulation provides a prolonged duration of efficacy when applied local into a tissue area in a subject in need thereof. These sustained release formulations are useful in treating localized pain in a subject in need thereof.

(I) Sustained Release Formulations

The present disclosure encompasses sustained release formulations. These sustained release formulations comprise (a) one or more active pharmaceutical ingredient(s); (b) at least one biocompatible polymer excipient; and (c) at least one biocompatible solvent wherein at least one active pharmaceutical ingredient has a particle size distribution ranging from about 0.5 μm to about 100.0 μm.

(a) One or More Active Pharmaceutical Ingredient(s)

The sustained release formulation comprises one or more active pharmaceutical ingredient(s). One of the active pharmaceutical ingredients in the sustained release formulation has a particle size distribution ranging from about 0.5 μm to about 100.0 μm.

The one or more active pharmaceutical ingredient(s) is an anesthetic drug, an anti-inflammatory drug (steroidal or non-steroidal), an antiemetic drug, or a combination thereof. In general, the one or more active ingredient(s) comprise bupivacaine, ropivacaine, levobupivacaine, lidocaine, buprenorphine, celecoxib, meloxicam, dexamethasone, betamethasone, betamethasone-21-acetate, triamcinolone acetonide, nepafenac, aprepitant, cox 1 inhibitors, cox 2 inhibitors, rolapitant, fosaprepitant, granisetron, ondansetron, palonosetron, prochlorperazine, hyaluronic acid, sodium hyaluronate, cross-linked derivatives of hyaluronic acid, or a combination thereof.

Generally, one of these active pharmaceutical ingredients has a particle size distribution ranging from about 0.5 μm to about 100.0 μm. In various embodiments, one of these one of these active pharmaceutical ingredients has a particle size distribution ranging from about 0.5 μm to about 100.0 μm, from about 5 μm to about 75 μm, from about 5 μm to about 50 μm, or from about 5 μm to about 15 μm including all subranges in between.

In general, the one or more active pharmaceutical ingredient(s) ranges from about 0.01 wt % to about 20.0 wt % (w/w of the total sustained release formulation). In various embodiments, the one or more active pharmaceutical ingredient(s) has a weight % of the total weight of the formulation which ranges from 0.01 wt % to about 20.0 wt %, from about 1.0 wt % to about 15.0 wt %, from about 2.5 wt % to about 10.0 wt %, or from 5.0 wt % to about 7.5 wt % including all subranges in between.

(b) At Least One Biocompatible Polymer Excipient

The sustained release formulation comprises at least one biocompatible polymer excipient. Non-limiting examples of suitable biocompatible polymer excipients may be hyaluronic acid, sodium hyaluronate, cross-linked derivatives of hyaluronic acid, PEG 3350, PEG 4000, polyethylene oxide (PolyOX), methylcellulose, hydroxypropyl methylcellulose, collagen, carboxymethyl cellulose, or a combination thereof.

Generally, the at least one biocompatible polymer excipient ranges from about 0.01 wt % to about 20.0 wt % (w/w of the total sustained release formulation). In various embodiments, the at least biocompatible polymer excipient ranges from about 0.01 wt % to about 20.0 wt %, from about 1.0 wt % to about 15.0 wt %, or from about 5.0 wt % to about 10.0 wt % including all subranges in between.

(c) At Least One Biocompatible Solvent

The sustained release formulation comprises at least one biocompatible solvent. Non-limiting examples of the at least one solvent may be PEG 200, PEG 300, PEG 400, EtOH, water, polysorbate 20, polysorbate 80, propylene glycol, NMP, DMSO, benzyl alcohol, glycerol, or a combination thereof.

In general, the at least one biocompatible solvent range from about 5.0 wt % to about 90.0 wt % (w/w of the total sustained release formulation). In various embodiments, the at least one biocompatible solvent range from about 5.0 wt % to about 90.0 wt %, from about 10.0 wt % to about 75 weight %, or from about 20.0 wt % to about 50.0 wt % including all subranges in between.

(d) Properties of the Sustained Release Formulation

The sustained release formulation, as detailed herein, exhibits various unique properties. The sustained release formulation exists as a suspension, a viscous mixture, or a gel. This sustained release formulation suspension is a partial gel of the one or more active pharmaceutical ingredient(s) and the at least one biocompatible polymer excipient due to the particle size distribution of the one or more active pharmaceutical ingredient(s). Upon contact with water or a physiological fluid (such as blood), the partial gel interacts with the water or the physiological fluid forming a gel. This in-situ gel provided the sustained release aspects of the formulation.

The sustained release formulation, after administration, provides a duration of release of the one or more active pharmaceutical ingredient(s) which is at least 2 times greater than the direct release formulation of the one or more active pharmaceutical ingredient(s). In various embodiments, the sustained release formulation provides a duration of release of one or more active pharmaceutical ingredient(s) which is at least 2 times greater, at least 3 times greater, at least 4 times greater, at least 5 times greater, at least 6 times greater, at least 7 times greater, at least 8 times greater, at least 9 times greater, or at least 10 times greater, as compared to the direct formulation of the one or more active pharmaceutical ingredient(s).

(II) Methods for Preparing the Sustained Release Formulation

Another aspect of the present disclosure encompasses a method for preparing the sustained release formulation. The method comprises contacting one or more active pharmaceutical ingredient(s), at least one biocompatible polymer excipient, and at least one solvent.

A list of suitable one or more active pharmaceutical ingredient(s) is detailed above in Section I(a). A list of at least one biocompatible polymer excipient and at least one solvent is detailed above in Section I(b) and Section I(c) respectively.

The components of the formulation comprising one or more active pharmaceutical ingredient(s), at least one biocompatible polymer excipient, and at least one biocompatible solvent may be added stepwise, in any sequential order, or all at once in a reaction vessel or reactor. In one embodiment, one of the active pharmaceutical ingredients is contacted and mixed with at least one biocompatible polymer excipient. The combination of the one active pharmaceutical ingredient and at least one biocompatible polymer excipient is then contacted and mixed with at least one biocompatible solvent to form suspension, a viscous mixture, or a gel.

Before initiation of the method, one or more of the active pharmaceutical ingredient(s) is micronized to a particle size distribution ranging from about 0.5 μm to about 100.0 μm. Non-limiting methods for micronizing the one or more pharmaceutical ingredient(s) may be jet milling, grinding, ball-milling, or homogenizing.

The temperature of contacting and mixing to prepare the sustained release formulation can and will vary depending on the specific one or more active pharmaceutical ingredient(s), the specific at least one biocompatible polymer excipient, the specific at least one solvent, and the amounts of each of these components. Generally, the temperature of contacting and mixing may range from 10° C. to about 40° C. In various embodiments, the temperature of contacting and mixing may range from 10° C. to about 40° C., from about 15° C. to about 35° C., or from about 20° C. to about 30° C. In one embodiment, the temperature of contacting and mixing may be at room temperature (˜23° C.).

As appreciated by the skilled artisan, the duration of mixing the components of the sustained release formulation is dependent on not only the components but also when the components are adequately dispersed and form a suspension, a viscous mixture, or a gel. In general, the duration of mixing can range from about 5 minutes to about an hour. In various embodiments, the duration of mixing can range from about 5 minutes to about an hour, from about 15 minutes to about 45 minutes, or from about 25 minutes to about 35 minutes.

After formation of the sustained release formulation, the formulation is stored at or below room temperature. This sustained release formulation can be stored for at least 2 years.

This sustained release formulation suspension is a partial gel of the one or more active pharmaceutical ingredient(s), the at least one biocompatible polymer excipient, and the at least one biocompatible solvent due to the particle size distribution of the one or more active pharmaceutical ingredient(s). Upon contact with water or a physiological fluid (such as blood), the partial gel interacts with the water or the physiological fluid forming an in-situ gel. This in-situ gel provided the sustained release aspects of the formulation.

(III) Methods of Treating Localized Pain in a Subject in Need

In yet another aspect, provides a method of treating localized pain in a subject in need, the method comprises locally administering the sustained release formulation as described in Section (I).

Without being bound to any theory, the formulations provide a method for treating localized pain. Upon administration of the partial gel or suspension through subcutaneous, intramuscular, injection into soft tissue, or injection into a joint cavity, these formulations initially contact water or a physiological fluid. Upon contact, these partial gels form a gelling delivery matrix. This in-situ gelling matrix provides an extended and sustained release of the one or more active pharmaceutical ingredient(s). These formulations can be used to treat localized pain post operatively, nausea, and vomiting (surgery, radiation, local chemotherapy).

Suitable subjects may include, without limit, humans, as well as companion animals such as cats, dogs, rodents, and horses; research animals such as rabbits, sheep, pigs, dogs, primates, mice, rats and other rodents; agricultural animals such as cows, cattle, pigs, goats, sheep, horses, deer, chickens and other fowl; zoo animals; and primates such as chimpanzees, monkeys, and gorillas. The subject can be of any age without limitation. In a preferred embodiment, the subject may be a human.

### Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above-described methods without departing from the scope of the invention, it is intended that all matter contained in the above description and in the examples given below, shall be interpreted as illustrative and not in a limiting sense.

### Example 1: Sample Preparation with Micronized Active Pharmaceutical Ingredient(s) (API) and Sodium Hyaluronate

Micronized levobupivacaine was prepared using a high-speed grinder. The desired API particle size was achieved by altering the grinder speed. The API can also be micronized using other instruments such as jet mill, homogenizer, or ball mill, etc. The micronized API and sodium hyaluronate, were mixed thoroughly and the powder blend was mixed with PEG 300 solution to form flowable or viscous cream-like suspension, according to the formulation composition. In some formulation's other active ingredients such as betamethasone-21-acetate was added to enhance the duration of action. The compositions of formulations were listed in the following Table 1.

TABLE 1
Composition of Formulations 1 to 8

| | | API | | | | | |
|---|---|---|---|---|---|---|---|
| | | PSD | | | Sodium | PEG | |
| Formulation | API | (um, | | API-2 | Hyaluronate | 300 | Water |
| ID | (mg/g) | Dv50) | API-2 | (mg/g) | (mg/g) | (mg/g) | (mg/g) |
| Formulation 1 | 25.9 | 17 | NA | 0.0 | 34.6 | 680.3 | 259.2 |
| Formulation 2 | 13.4 | 17 | NA | 0.0 | 17.8 | 701.6 | 267.3 |
| Formulation 3 | 27.2 | 9 | NA | 0.0 | 36.3 | 678.2 | 258.4 |
| Formulation 4 | 47.0 | 9 | Betamethasone- | 1.48 | 31.3 | 666.3 | 253.8 |
| | | | 21-acetate | | | | |
| Formulation 5 | 26.4 | 9 | Betamethasone- | 0.88 | 17.6 | 691.7 | 263.5 |
| | | | 21-acetate | | | | |
| Formulation 6 | 27.8 | 6.9 | NA | 0.0 | 55.6 | 663.7 | 252.8 |
| Formulation 7 | 27.2 | 6.9 | NA | 0.0 | 72.6 | 680.0 | 220.2 |
| Formulation 8 | 29.9 | 6.2 | NA | 0.0 | 39.9 | 673.6 | 256.6 |

After preparation, the assay, invitro release of formulations were tested. The anesthesia efficacy of some formulations was also evaluated in animal models.

### Example 2: Preparation of Formulations Using Micronized API and PolyOX

Different polymers can be used to prepare the formulations. Micronized levobupivacaine was mixed thoroughly with PolyOX. The powder blend was mixed with PEG 300 solution to form uniform suspension. The composition of 4 formulations were listed in the following Table 2.

TABLE 2
Composition of Formulations 9 to 12

| | | | | | PEG | |
|---|---|---|---|---|---|---|
| | API | PolyOX | PolyOX | Tween 20 | 300 | Water |
| Formulation ID | (mg/g) | Grade | (mg/g) | (mg/g) | (mg/g) | (mg/g) |
| Formulation 9 | 26.8 | 0.0 | 0.0 | 0.55 | 704.7 | 268.5 |
| Formulation 10 | 26.1 | 1125 | 26.1 | 0.55 | 686.3 | 261.4 |
| Formulation 11 | 26.1 | 301 | 26.1 | 0.55 | 686.3 | 261.4 |
| Formulation 12 | 26.1 | 303 | 26.1 | 0.55 | 686.3 | 261.4 |

After preparation, the assay and in-vitro release of the formulations were tested.

TABLE 3
Formulations 13 to 21

| | | | | | Sodium | | Solvent | | Solvent | |
|---|---|---|---|---|---|---|---|---|---|---|
| Formulation | | API-1 | | API-2 | hyaluronate | Solvent | A | Solvent | B | Water |
| ID | API-1 | (mg/g) | API-2 | (mg/g) | (mg/g) | A | (mg/g) | B | (mg/g) | (mg/g) |
| Formulation | LBP | 27.3 | NA | 0 | 27.3 | PEG | 564.0 | Ethanol | 107.9 | 273.4 |
| 13 | | | | | | 300 | | | | |
| Formulation | LBP | 27.0 | NA | 0 | 72.1 | PEG | 737.1 | NA | 0 | 163.8 |
| 14 | | | | | | 300 | | | | |
| Formulation | RP | 26.4 | NA | 0 | 17.6 | PEG | 692.3 | NA | 0 | 263.7 |
| 15 | | | | | | 300 | | | | |
| Formulation | LBP | 26.3 | Meloxicam | 1.32 | 17.6 | PEG | 691.4 | NA | 0 | 263.4 |
| 16 | | | | | | 300 | | | | |
| Formulation | LBP | 28.3 | Nepafenac | 1.42 | 37.7 | PEG | 675.3 | NA | 0 | 257.3 |
| 17 | | | | | | 300 | | | | |
| Formulation | LBP | 27.2 | Triamcinolone | 3.33 | 72.6 | PEG | 649.5 | NA | 0 | 247.4 |
| 18 | | | acetonide | | | 300 | | | | |
| Formulation | LBP | 27.3 | NA | 0 | 72.8 | PEG | 651.6 | NA | 0 | 248.2 |
| 19 | | | | | | 400 | | | | |
| Formulation | LBP | 27.3 | NA | 0 | 72.8 | NMP | 651.6 | NA | 0 | 248.2 |
| 20 | | | | | | | | | | |
| Formulation | LBP | 27.3 | NA | 0 | 72.8 | DMSO | 651.6 | NA | 0 | 248.2 |
| 21 | | | | | | | | | | |

API-1. LBP: levobupivacaine; RP: ropivacaine.

After preparation, these formulations were tested for assay and in-vitro release. Selected formulations were evaluated for efficacy in animal models.

### Example 4: In-Vitro Release of Formulations

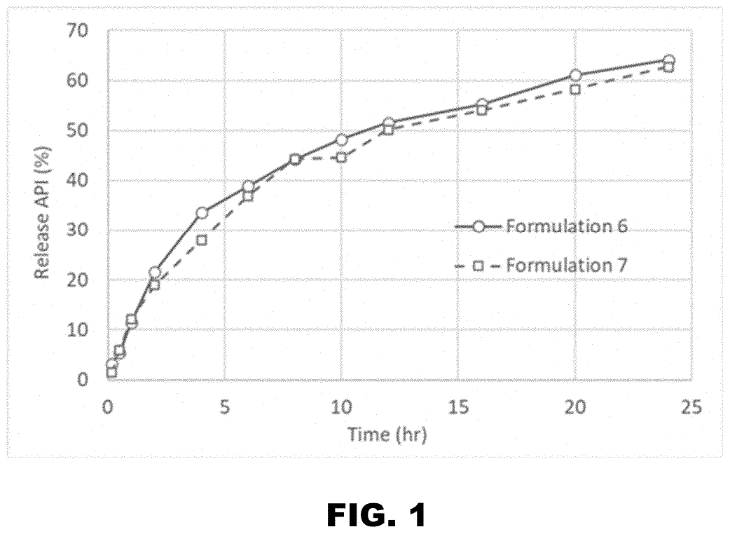

The in-vitro release of API from formulation 6 and 7 were tested using USP dissolution apparatus, type 2. One gram of each formulation was carefully loaded into dialysis cellulose membrane column (Float-A-Lyzer G2, 1000Kd MWCO). The dialysis columns were then installed on disso Agilent 708-DS and placed in 1000 ml buffer (pH6.6, phosphate-citrate buffer). The buffer temperature was maintained at 37° C. during in-vitro release test and paddle was stirred at 100 rpm. At each desired time point, 2 ml buffer was transferred and the content of levobupivacaine was analyzed by HPLC. The result was plotted in FIG. 1. The in-vitro release (IVR) profiles of Formulation 6 and 7 are comparable though the hyaluronate content in these formulations was different.

### Example 5: Evaluation of In Situ Forming Hydrogels by In-Vitro Release Testing

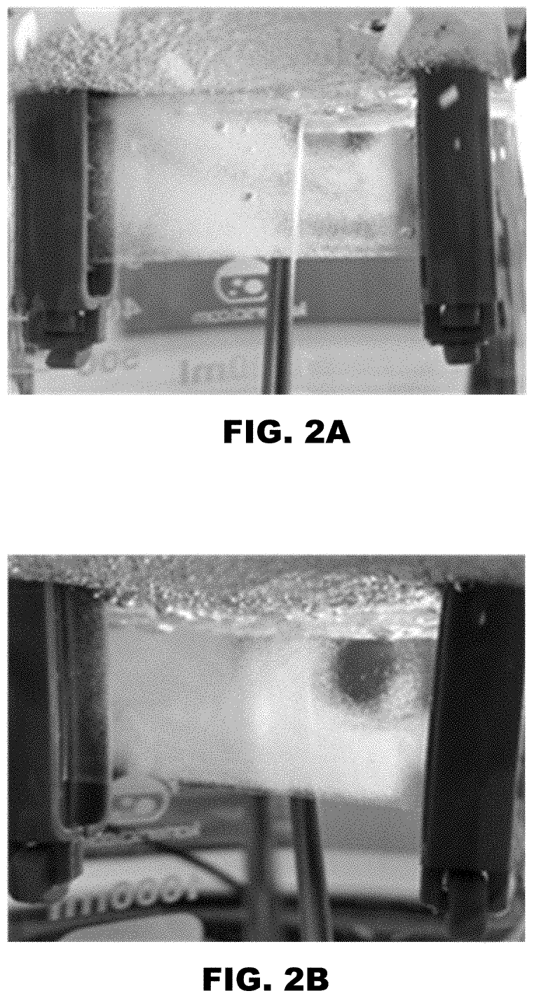

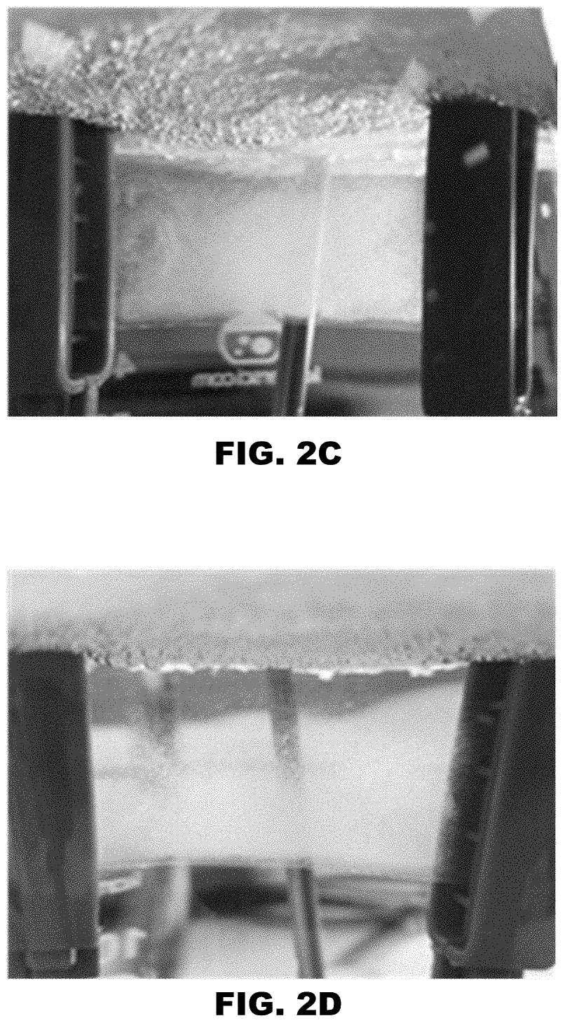

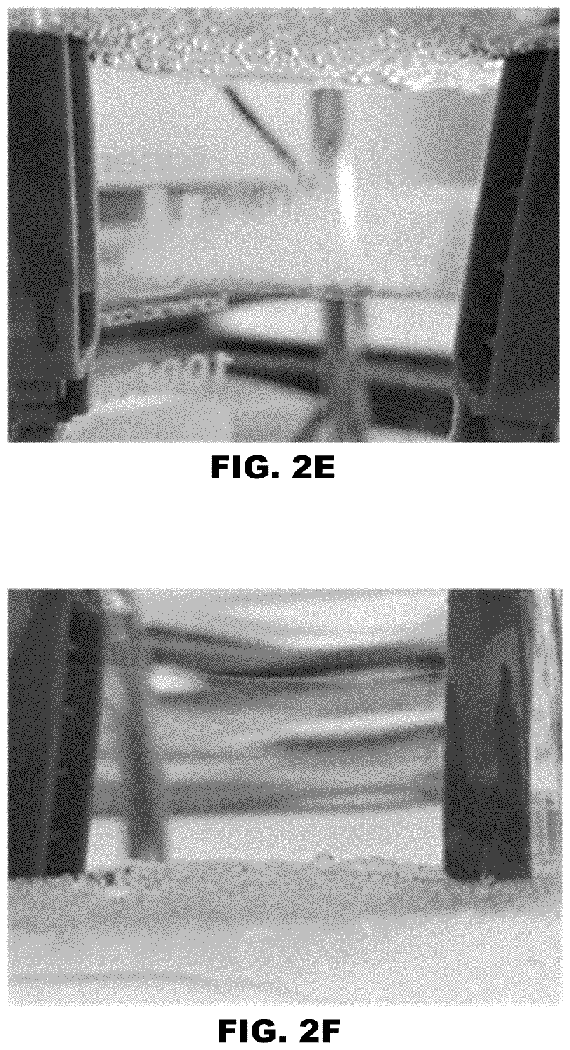

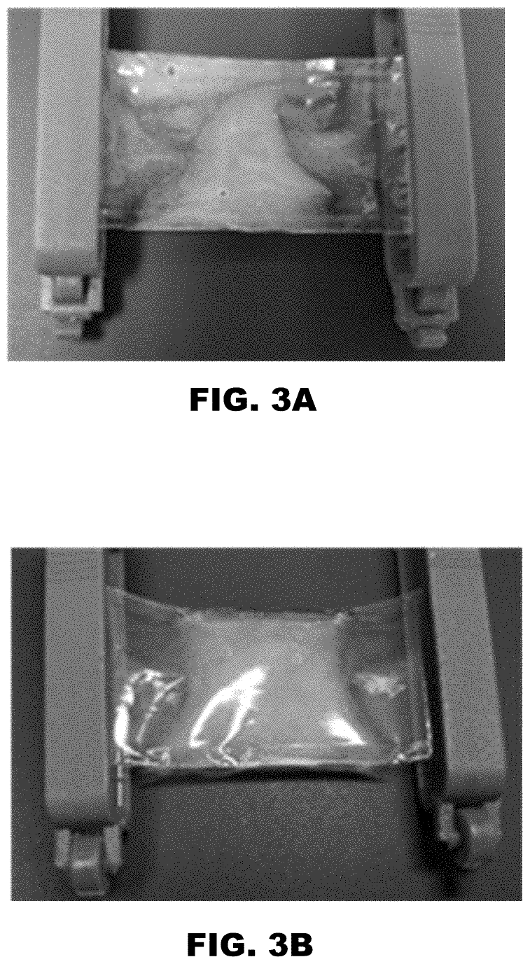

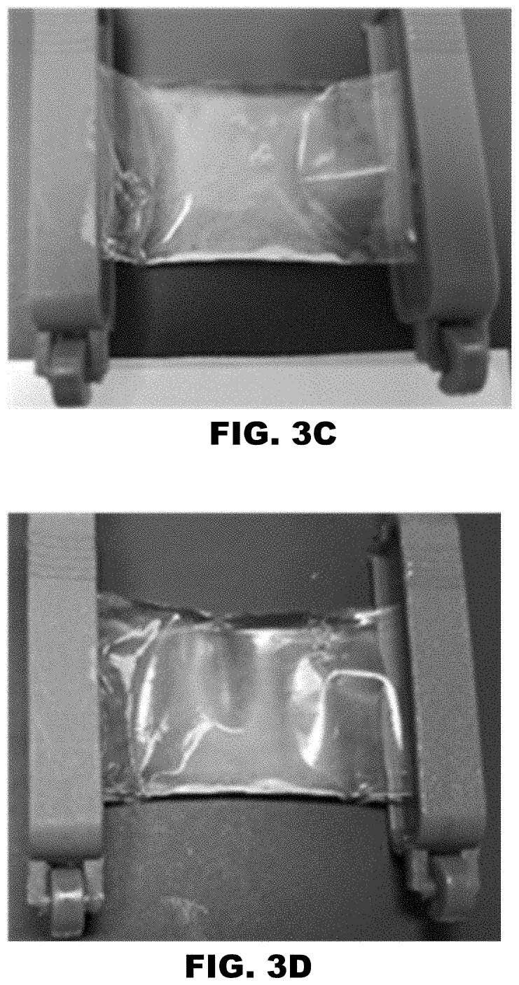

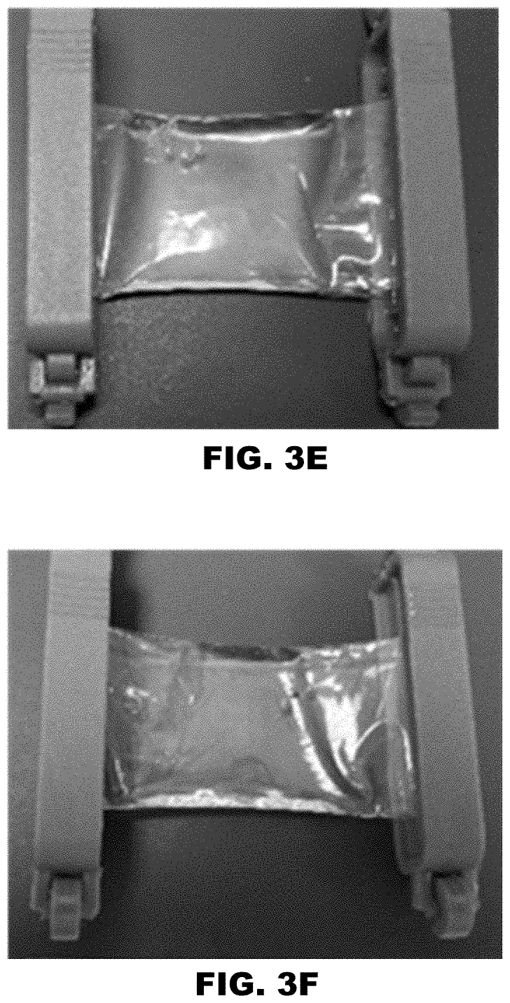

The release rate of drug is correlated to the formation of the hydrogel, and the interaction of drug with hydrogel polymer. To evaluate the hydrogel formation and drug release, an in-vitro release study was performed to monitor the gelling and drug release process. The formulation 8 was loaded inside the 33×60 mm cellulose dialysis tubing and sealed with dialysis tubing clamps. The clamps were hold through the holes in the floatation ring. The dialysis tubing was floated in the dialysate reservoir containing a stir bar and adjust the stirring rate to form a gentle rotating current. The samples were dialyzed at 37° C. with surfactants in the phosphate buffer solutions. In-process analysis was done by removing a small amount of solution periodically from the dialysis reservoir. The dialysis tubing was also removed from the reservoir to take a picture and measure the total weight. Representative pictures of the dialysis tubing are shown in FIG. 2A-2F and FIG. 3A-3F. The net weight changes are summarized in Table 4.

TABLE 4
Net weight and changes at different time points

| Time (hr) | Weight (g) | Net weight (g) |
|---|---|---|
| 0 | 13.28 | 0.00 |
| 1 | 16.42 | 3.14 |
| 2 | 17.14 | 3.86 |
| 4 | 17.36 | 4.08 |
| 6 | 17.52 | 4.25 |
| 24 | 17.55 | 4.27 |

The weight gain of formulation in the dialysis bag during in-vitro release test is shown in Table 4 also indicated the gelation of sodium hyaluronate over time.

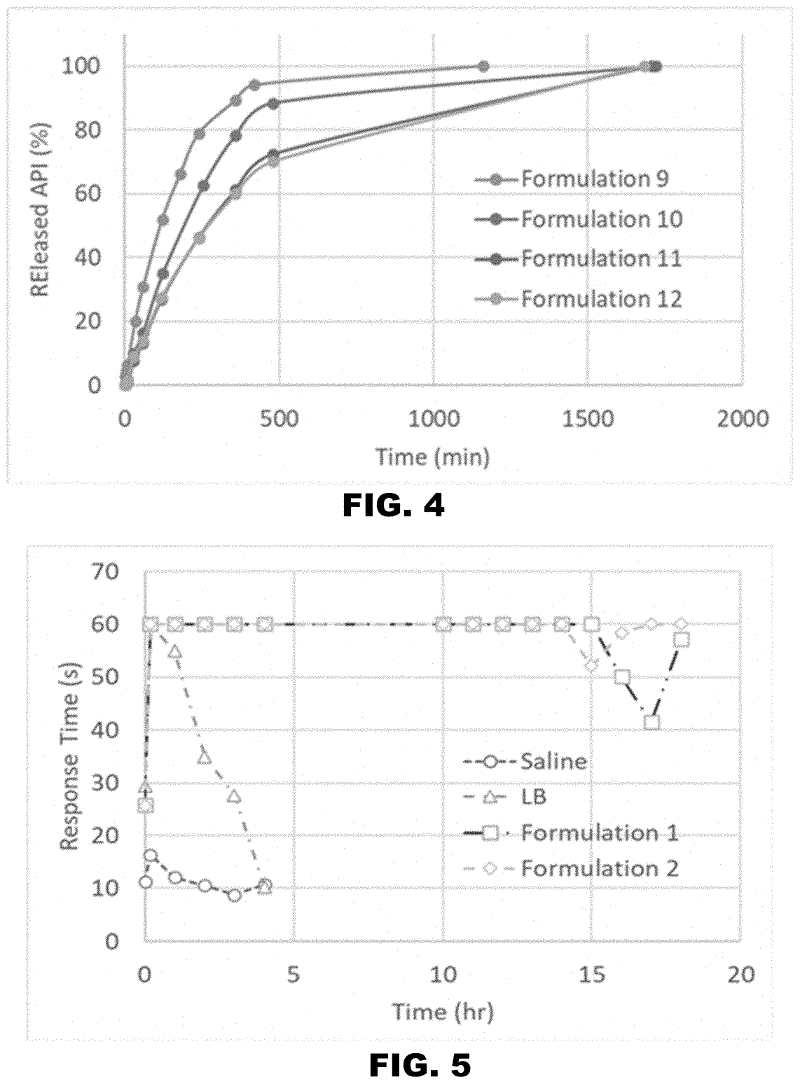

The in-vitro release of API from formulation 9, 10, 11 and 12 was also tested. One gram of each formulation was carefully loaded into dialysis cellulose membrane tubing (Sigma, 50 k MWCO). The dialysis membrane tubing was closed by two dialysis tubing clamps and placed in 1000 ml buffer (pH6.8, 1.0% Brij). The buffer temperature was maintained at 37 C during in-vitro release test and stirred at 100 rpm by a magnetic stirrer. At each desired time point, 1 ml buffer was transferred and the content of levobupivacaine was analyzed by HPLC. The IVR results of formulations were depicted in FIG. 4. The results showed that the addition of PolyOX slowed down the drug release from the formulations. The drug release rate is slower in formulations with higher molecular weight PolyOX compared to the formulations with low molecular weight PolyOX.

### Example 6: Animal Study, Rat Sciatic Nerve Block Hotplate Pain Model

Rat sciatic nerve block model was used to evaluate the anesthetic efficacy of formulations. Young adult male Sprague-Dawley rats (180-220 g) were housed in groups of 4 per cage with rat food and water ad libitum. The animal living room was controlled at 23° C. with a 12 hours light/12 hours dark circadian cycle. A needle was introduced posteromedial to the area of the popliteal fossa, and 0.3-1.0 mL of the test sample was injected once bone was contacted, depositing the injectate over the sciatic nerve. The test samples were injected into both hind limbs.

Thermal nociception was assessed using a hotplate test. Animals were exposed to a 50° C. hot plate. The time (latency) until paw withdrawal and lick was measured with a stopwatch. If the animal did not lick its paw within 60 seconds, then the experimenter removed the rat from hot plate to prevent thermal injury or the development of hyperalgesia. Before administration all rats were tested on the hotplate twice to obtain the response baseline. The animals have too short response time (<5s) or too slow response (>40s) were removed. The qualified animals were then randomly divided into different groups, four animals in each group that has similar average response time. Four groups were injected with saline, levobupivacaine HCl, formulations 1 and 2, respectively. Hot plate testing was performed at the following intervals after injection: 10 min, 30 min, 60 min, then hourly until no anesthesia efficacy or up to 18 hours. The result is presented in the following FIG. 5.

The Formulation 1 and 2 showed extended efficacy compared to levobupivacaine HCl sample demonstrating superior efficacy of the suspension formulations. The drug concentration difference in these two formulations didn't affect the efficacy significantly.

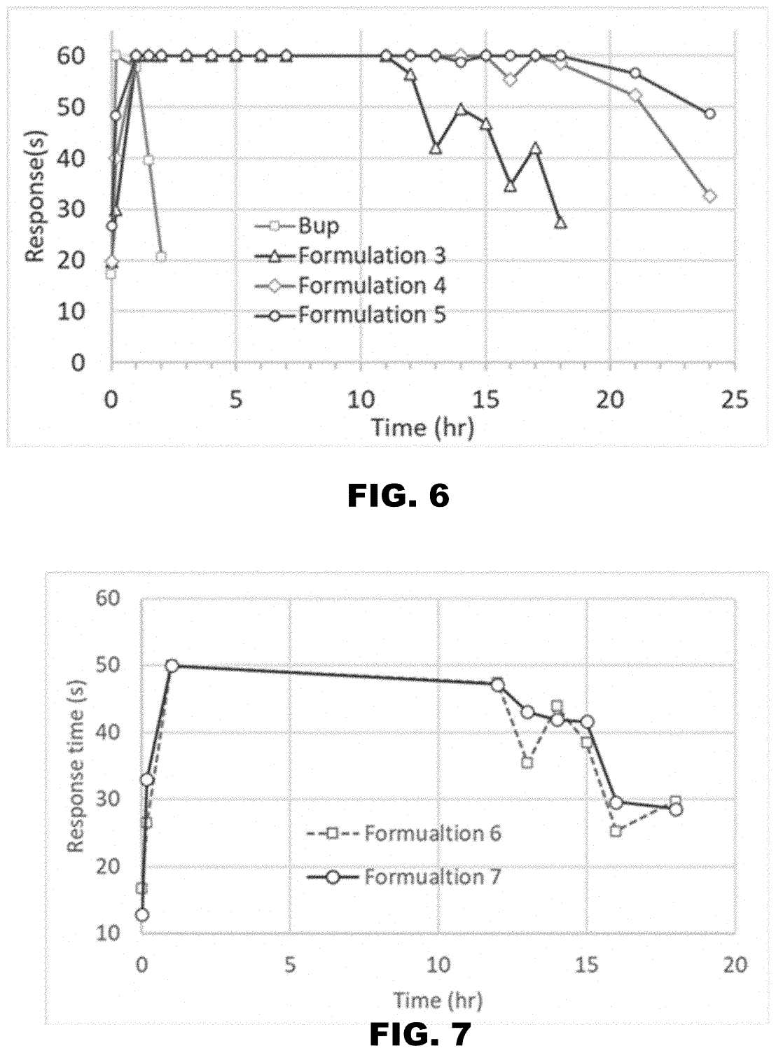

In another rat sciatic nerve block study, four groups of rats were injected with bupivacaine HCl, Formulation 3, Formulation 4, and Formulation 5. The hot-plate test lasted up to 24 hours. The result is presented in the following FIG. 6.

The Formulation 3 showed prolonged efficacy compared to bupivacaine HCl. The addition of betamethasone-21-acetate in formulation 4 and 5 further improved the efficacy period.

In another rat sciatic nerve block study, two groups of rats were injected with Formulation 6 and Formulation 7. To minimize the potential heat damage to rats' paw, the on-plate test time was set to 50 seconds. The result is presented in the following FIG. 7.

Both Formulation 6 and 7 showed similar efficacy period. The different amounts of sodium hyaluronate used in these two formulations didn't affect the efficacy significantly in rat sciatic block model.

### Example 7: Animal Study Mini-Pig Skin Incision Model

Mini-pig skin incision model was used to test the anesthetic efficacy of some formulations. Mini pig was used in this model due to the similarities of their skin to humans.

Mini pigs (9-12 kg) were randomly assigned for test groups. Under isoflurane anesthesia and sterile surgical conditions, a 6 cm long incision was made through skin in the rear left flank. The test drugs were administered subcutaneously into both sides of incision. The wound was then closed by continuous suture. After surgery the mini pig received antibiotic amoxicillin injection for 3 days as wound care.

The efficacy of test drugs was evaluated by Von Frey test. At desired time point, an electrical automatic Von Frey was used to apply force about 0.5 cm to the incision. If operator observed the contraction of skin/muscle, or the applied force was over 100 g, the operator stopped the test and record the read of applied force. The response baseline of all mini pigs

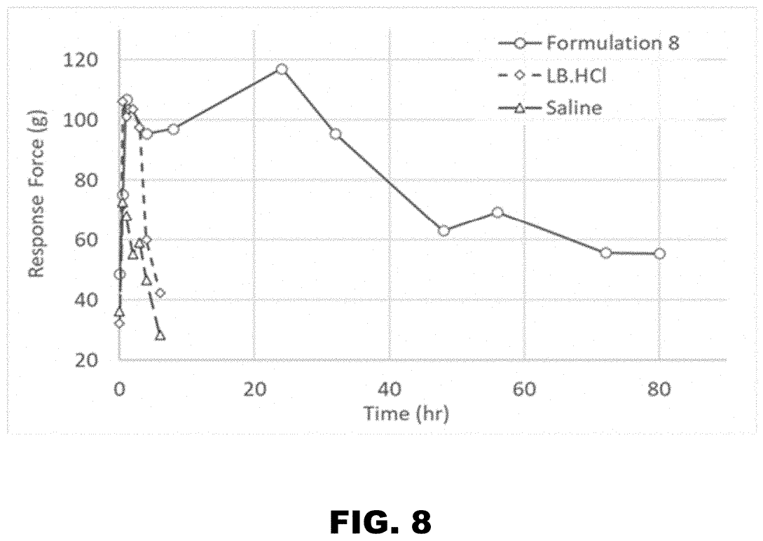

was tested and the pain threshold was set to the middle of baseline and 100 g. If the response force was higher than pain threshold value then there was anesthesia efficacy, vice versa. The anesthesia efficacy result of mini-pig incision model is showed in FIG. 8.

The mini-pigs injected with saline could feel the pain after 30 min of surgery. As the effect of isoflurane anesthesia subsided, the saline group mini-pigs' response force dropped dramatically. The efficacy of levobupivacaine HCl injection can last for about 4 hours, which is similar to reported literature. The anesthesia efficacy of Formulation 8 lasted over 40-56 hours, which is significantly longer than levobupivacaine HCl.

What is claimed is:

1. A sustained release formulation, the sustained release formulation comprises:
a. one or more active pharmaceutical ingredient(s);
b. at least one biocompatible polymer excipient; and
c. at least one biocompatible solvent;
wherein one of the active pharmaceutical ingredients has a particle size distribution ranging from about 0.5 μm to about 100.0 μm.

2. The sustained release formulation of claim 1, wherein the one or more pharmaceutical ingredient(s) is an anesthetic drug, an anti-inflammatory drug, an antiemetic drug, or a combination thereof.

3. The sustained release formulation of claim 2, wherein the one or more active pharmaceutical ingredient(s) comprise bupivacaine, ropivacaine, levobupivacaine, lidocaine, buprenorphine, celecoxib, meloxicam, dexamethasone, betamethasone, betamethasone-21-acetate, triamcinolone acetonide, nepafenac, aprepitant, cox 1 inhibitors, cox 2 inhibitors, rolapitant, fosaprepitant, granisetron, ondansetron, palonosetron, prochlorperazine, hyaluronic acid, sodium hyaluronate, cross-linked derivatives of hyaluronic acid, or a combination thereof.

4. The sustained release formulation of claim 1, wherein the at least one biocompatible polymer excipient comprises hyaluronic acid, sodium hyaluronate, cross-linked derivatives of hyaluronic acid, PEG 3350, PEG 4000, polyethylene oxide (PolyOX), methylcellulose, hydroxypropyl methylcellulose, collagen, carboxymethyl cellulose, or a combination thereof.

5. The sustained release formulation of claim 1, wherein the at least one biocompatible solvent comprises PEG 200, PEG 300, PEG 400, EtOH, water, polysorbate 20, polysorbate 80, propylene glycol, NMP, DMSO, benzyl alcohol, glycerol, or a combination thereof.

6. The sustained release formulation of claim 1, wherein the sustained release formulation is a suspension, a viscous mixture, or a gel.

7. The sustained release formulation of claim 6, wherein the sustained release formulation is a partial gel of the one or more active pharmaceutical ingredient(s), the at least one biocompatible polymer excipient, and the at least one biocompatible solvent.

8. The sustained release formulation of claim 7, wherein the sustained release formulation forms an in-situ gel upon contact with water or physiological fluid.

9. The sustained release formulation of claim 1, wherein the one or more active pharmaceutical ingredient(s) ranges from about 0.01 wt % to about 20.0 wt % (w/w of the total sustained release formulation).

10. The sustained release formulation of claim 1, wherein the at least one biocompatible polymer excipient ranges from about 0.01 wt % to about 20.0 wt % (w/w of the total sustained release formulation).

11. The sustained release formulation of claim 1, wherein the at least one biocompatible solvent ranges from about 5.0 wt % to about 90.0 wt % (w/w of the total sustained release formulation).

12. A method of preparing a sustained release formulation, the method comprises contacting one or more active pharmaceutical ingredient(s), at least one biocompatible polymer excipient, and at least one biocompatible solvent; wherein one of the active pharmaceutical ingredients has a particle size distribution ranging from about 0.5 μm to about 100.0 μm.

13. The method of claim 12, wherein the one or more active pharmaceutical ingredient(s), the at least one biocompatible polymer excipient, and the at least one solvent may be added stepwise, in any sequential order, or all at once.

14. The method of claim 12, wherein the active pharmaceutical ingredient(s) comprises bupivacaine, ropivacaine, levobupivacaine, lidocaine, buprenorphine, celecoxib, meloxicam, dexamethasone, betamethasone, betamethasone-21-acetate, triamcinolone acetonide, nepafenac, aprepitant, cox 1 inhibitors, cox 2 inhibitors, rolapitant, fosaprepitant, granisetron, ondansetron, palonosetron, prochlorperazine, hyaluronic acid, sodium hyaluronate, cross-linked derivatives of hyaluronic acid, or a combination thereof.

15. The method of claim 12, wherein the at least one biocompatible polymer excipient comprises hyaluronic acid, sodium hyaluronate, cross-linked derivatives of hyaluronic acid, PEG 3350, PEG 4000, polyethylene oxide (PolyOX), methylcellulose, hydroxypropyl methylcellulose, collagen, carboxymethyl cellulose, or a combination thereof.

16. The method of claim 12, wherein the at least one biocompatible solvent comprises PEG 200, PEG 300, PEG 400, EtOH, water, polysorbate 20, polysorbate 80, propylene glycol, NMP, DMSO, benzyl alcohol, glycerol, or a combination thereof.

17. The method of claim 12, wherein the sustained release formulation is a suspension, a viscous mixture, or a gel.

18. A method of treating localized pain in a subject in need, the method comprises locally administering the sustained release formulation of claim 1.

19. The method of claim 18, wherein the subject is a human or a non-human animal.

20. The method of claim 18, wherein the sustained release formulation, after administration, form in-situ gel upon contact with water or physiological fluid.
